(19) United States
(12) Patent Application Publication
(10) Pub. No.: US 2012/0310094 A1
(43) Pub. Date: Dec. 6, 2012

(54) ULTRASOUND DIAGNOSTIC APPARATUS

(75) Inventors: Yukiya MIYACHI, Ashigara-kami-gun (JP); Masafumi NOGUCHI, Ashigara-kami-gun (JP)

(73) Assignee: FUJIFILM CORPORATION, Tokyo (JP)

(21) Appl. No.: 13/485,386

(22) Filed: May 31, 2012

(30) Foreign Application Priority Data
Jun. 3, 2011 (JP) 2011-125354

(51) Int. Cl. A61B 8/02; A61B 8/08; A61B 8/14

(52) U.S. Cl. 600/443

(57) ABSTRACT

An ultrasound diagnostic apparatus adapted for vascular wall elastic modulus measurement comprises an ultrasound probe, an actuation controller, an image producer, a region-of-interest setter, and a storage unit. The apparatus sets a region of interest, incorporates ultrasonic wave transmission/reception for obtaining an ultrasound image including the region of interest into ultrasonic wave transmission/reception for obtaining an ultrasound image of the region of interest at a predetermined timing, and stores the obtained two ultrasound images in association with each other, which makes the region of interest easier to find in an ultrasound image for diagnosis, and allows a satisfactory follow-up.

### BACKGROUND OF THE INVENTION

The present invention relates to an ultrasound diagnostic apparatus which is suitable for measuring an elastic modulus of a vascular wall, and in particular, to an ultrasound diagnostic apparatus which facilitates detection of a blood vessel anterior wall boundary from a B-mode image.

An ultrasound diagnostic apparatus using an ultrasound image has hitherto been put into practical use in the field of medicine.

In general, this type of ultrasound diagnostic apparatus has an ultrasound probe (hereinafter, referred to as a probe) and a diagnostic apparatus body. Ultrasonic waves are transmitted from the probe toward a subject, an ultrasonic echo from the subject is received by the probe, and a reception signal is electrically processed by the diagnostic apparatus body to produce an ultrasound image.

Ultrasonic waves are transmitted toward a blood vessel, a cardiac wall, or the like, an ultrasonic echo therefrom is received, and a reception signal is analyzed to obtain the displacement of a vascular wall or the like. The elastic modulus of the vascular wall, the cardiac wall (heart muscle), or the like is measured from the displacement.

For example, JP 10-5226 A describes a technique in which ultrasonic waves are transmitted and received with respect to an object moving in synchronization with the heartbeats (cardiac pulsation) to obtain a reception signal of an ultrasonic echo, the instantaneous position of the object is determined using the amplitude and phase of the reception signal, and the large-amplitude displacement motion of the vascular wall based on the heartbeats is tracked, thereby obtaining the elastic modulus of the blood vessel.

Specifically, the motion velocity waveform of minute vibration of the vascular wall is obtained on the basis of the sequential position of the vascular wall, the tracking trajectory of each of the sections at a predetermined interval in the depth direction in the vascular wall is obtained, and a temporal change in thickness of each section is calculated to obtain the elastic modulus of the blood vessel.

Similarly, JP 2010-233956 A describes an ultrasound diagnostic apparatus which obtains the displacement of a blood vessel or the like from a reception signal of an ultrasonic echo obtained when ultrasonic waves are transmitted and received with respect to an object moving in synchronization with the heartbeats, and obtains an elastic modulus from the displacement.

In this ultrasound diagnostic apparatus, a B-mode image and an M-mode image are produced using a reception signal obtained from an object, such as a blood vessel. Blurring due to hand or body movement is detected from the reception signal of the M-mode image, and the positional variation of the probe and the subject is detected using the reception signal of the M-mode image where the blurring is detected. The accuracy of the reception signal is determined from the detection result, and the displacement of the object is obtained using the reception signal of the M-mode image whose accuracy is determined to be high, and the elastic modulus of the vascular wall or the like is measured from the displacement.

### SUMMARY OF THE INVENTION

With regard to the measurement of the blood vessel elastic modulus using the ultrasound diagnostic apparatus or the measurement of an intima-media thickness (IMT) which has been heretofore used in the diagnosis of arteriosclerosis, in order to do follow-up, it is preferable to make measurement at the same position of the blood vessel at the time of a previous measurement and a subsequent measurement.

To this end, it is necessary that the position of a previous measurement made in the blood vessel can be easily found.

In general, the measurements of the blood vessel elastic modulus and the IMT are made using the ultrasound image of a vascular wall in the vicinity of the carotid sinus. Accordingly, the position of a place (vascular wall) where measurement has been made is stored on the basis of the distance from the carotid sinus, thereby finding a previous measurement position.

In order to make the measurement of the IMT with high precision, it is necessary to enlarge an image. In order to obtain the blood vessel elasticity index, such as the blood vessel elastic modulus, from the viewpoint of not only a high-precision measurement but also the maintenance of a high frame rate corresponding to the heartbeats, it is necessary to enlarge an image.

For this reason, the measurements are made by setting a region of interest (ROI) at the position of the blood vessel in the B-mode image, displaying the ROI on an enlarged scale, and analyzing the B-mode image of the ROI displayed on an enlarged scale or the M-mode image of a display line set in the B-mode image of the ROI.

In the conventional ultrasound diagnostic apparatus, display is performed on an enlarged scale. Accordingly, even if the distance from the carotid sinus is stored, there are many cases where it is difficult to accurately find where a previous measurement has been made in the blood vessel, making it difficult to do follow-up appropriately.

The invention has been accomplished in order to solve the problems with the prior art, and an object of the invention is to provide an ultrasound diagnostic apparatus capable of accurately and easily finding where a previous measurement has been made in the blood vessel at the time of the measurement of the blood vessel elastic modulus, the measurement of the IMT, or the like.

In order to achieve the above object, the present invention provides an ultrasound diagnostic apparatus comprising: an ultrasound probe which has ultrasound transducers transmitting ultrasonic waves, receiving an ultrasonic echo reflected by a subject, and outputting a reception signal according to the received ultrasonic echo; an actuation controller for controlling transmission/reception of ultrasonic waves by the ultrasound transducers; an image producer for producing an ultrasound image from the reception signal output from the ultrasound transducers; a region-of-interest setter for setting a region of interest in the ultrasound image of a predetermined size produced by the image producer; and a storage unit for storing the ultrasound image produced by the image producer, wherein: after the region of interest is set by the region-of-interest setter, the actuation controller controls transmission/reception of ultrasonic waves by the ultrasound transducers such that region-of-interest transmission/reception which is transmission/reception of ultrasonic waves corresponding to the region of interest is performed, and whole image transmission/reception for obtaining the ultrasound image of the predetermined size including the set region of interest is incorporated into the region-of-interest transmission/reception at a predetermined timing; after the region of interest is set by the region-of-interest setter, the image producer produces a region-of-interest image which is obtained by processing a reception signal by the region-of-interest transmission/reception and in which the region of interest is enlarged relative to the ultrasound image of the predetermined size, and the whole image which is the ultrasound image of the predetermined size obtained by processing a reception signal by the whole image transmission/reception; and the storage unit stores the region-of-interest image and the whole image including the region of the region-of-interest image in association with each other.

It is preferable that the ultrasound diagnostic apparatus as above further comprises a moving velocity detector for detecting a moving velocity of a vascular wall, and the actuation controller predicts a time, at which the moving velocity of the vascular wall is highest, in accordance with a detection result of the moving velocity of the vascular wall by the moving velocity detector, and incorporates the whole image transmission/reception into the region-of-interest transmission/reception when a predetermined period has elapsed from the predicted highest velocity time.

It is also preferable that the ultrasound diagnostic apparatus as above further comprises a heartbeat detector for detecting a heartbeat, and the actuation controller incorporates the whole image transmission/reception into the region-of-interest transmission/reception when a predetermined period has elapsed from a start of the heartbeat in accordance with a detection result of the heartbeat by the heartbeat detector.

The actuation controller preferably controls transmission/reception of ultrasonic waves by the ultrasound transducers such that a frame rate of the region-of-interest transmission/reception is higher than frame rates of ultrasonic wave transmission/reception for obtaining the ultrasound image of the predetermined size where the region of interest is set, and of the whole image transmission/reception.

Preferably, after the region of interest is set by the region-of-interest setter, the image producer produces a B-mode image of the region of interest as the region-of-interest image and an M-mode image of the region of interest from a reception signal by the region-of-interest transmission/reception, and produces a B-mode image of a predetermined size as the whole image from a reception signal by the whole image transmission/reception.

It is preferable that, after the region of interest is set by the region-of-interest setter, the region-of-interest image and the M-mode image of the region of interest produced by the image producer are displayed on a single screen.

It is also preferable that the region-of-interest image and the whole image including the region of the region-of-interest image are displayed on a single screen in accordance with an input instruction.

It is preferable that the ultrasound diagnostic apparatus further comprises an analyzer for analyzing an ultrasound image, and an analysis result by the analyzer is incorporated into the whole image and displayed.

Preferably, the whole image is an ultrasound image of the same size as an ultrasound image when the region of interest is set.

The ultrasound diagnostic apparatus of the invention configured as above sets a region of interest (ROI), when performing transmission/reception of ultrasonic waves for obtaining the ultrasound image of the ROI, performs transmission/reception of ultrasonic waves for obtaining an ultrasound image (whole image) of a predetermined size including the ROI at a predetermined timing, and stores the ultrasound image of the ROI and the whole image in association with each other.

For this reason, according to the ultrasound diagnostic apparatus of the invention, when newly measuring a blood vessel elastic modulus or the like of a patient who has been previously subjected to measurement of an elasticity index, such as a blood vessel elastic modulus, or IMT measurement, the ultrasound image of the ROI and the whole image stored are retrieved and displayed, and thus, it is possible to easily and accurately find the position of the blood vessel which has been previously measured, thereby enabling a new measurement. Therefore, according to the invention, it becomes possible to do follow-up satisfactorily, thereby making an appropriate diagnosis.

### BRIEF DESCRIPTION OF THE DRAWINGS

FIG. 1 is a diagram conceptually illustrating an example of an ultrasound diagnostic apparatus of the invention.

FIG. 2 is a block diagram conceptually illustrating the configuration of the ultrasound diagnostic apparatus illustrated in FIG. 1.

FIG. 3 is a flowchart for explaining an example of elasticity measurement of a vascular wall in the ultrasound diagnostic apparatus illustrated in FIG. 1.

FIG. 4 is a conceptual diagram for explaining an ultrasound diagnosis for elasticity measurement of a vascular wall.

FIGS. 5A and 5B are conceptual diagrams illustrating an example of image display in the ultrasound diagnostic apparatus illustrated in FIG. 1.

FIGS. 6A and 6B are conceptual diagrams illustrating an example of image display in the ultrasound diagnostic apparatus illustrated in FIG. 1.

FIGS. 7A to 7C are conceptual diagrams illustrating an example of image display in the ultrasound diagnostic apparatus illustrated in FIG. 1.

FIGS. 8A and 8B are conceptual diagrams illustrating an example of image display in the ultrasound diagnostic apparatus illustrated in FIG. 1.

FIG. 9 is a conceptual diagram illustrating an example of image display in the ultrasound diagnostic apparatus illustrated in FIG. 1.

FIGS. 10A to 10G are conceptual diagrams illustrating an example of image display in the ultrasound diagnostic apparatus illustrated in FIG. 1.

FIGS. 11A and 11B are conceptual diagrams illustrating an example of image display in the ultrasound diagnostic apparatus illustrated in FIG. 1.

FIG. 12 is a conceptual diagram illustrating an example of image display in the ultrasound diagnostic apparatus illustrated in FIG. 1.

FIG. 13 is a conceptual diagram illustrating an example of image display in the ultrasound diagnostic apparatus illustrated in FIG. 1.

### DETAILED DESCRIPTION OF THE INVENTION

Hereinafter, an ultrasound diagnostic apparatus of the invention will be described in detail on the basis of a preferred example illustrated in the accompanying drawings.

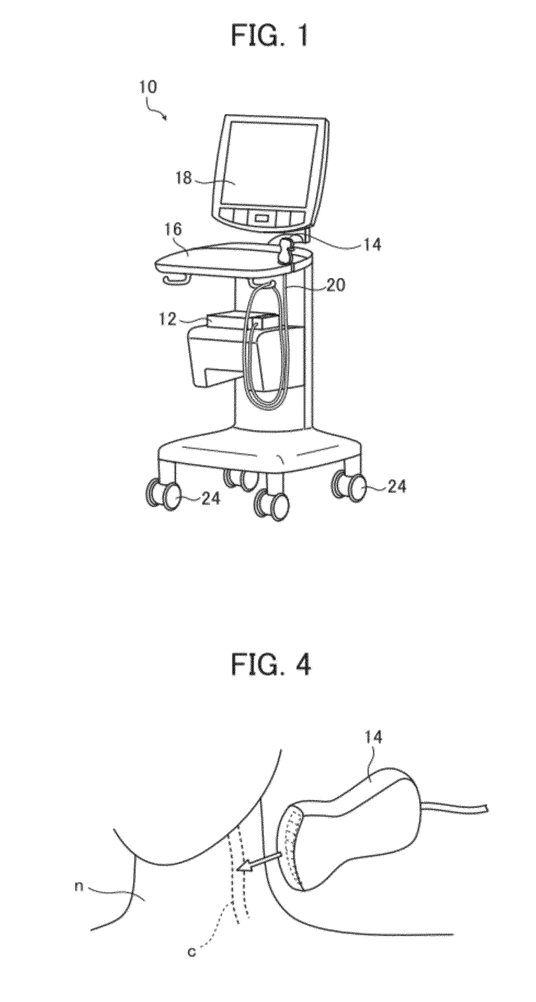

FIG. 1 conceptually illustrates the appearance of an example of the ultrasound diagnostic apparatus of the invention.

As illustrated in FIG. 1, an ultrasound diagnostic apparatus 10 basically has a diagnostic apparatus body 12, an ultrasound probe 14, an operating panel 16, and a display 18. Casters 24 are arranged at the lower end of the ultrasound diagnostic apparatus 10, such that the apparatus can be easily moved by human power.

The ultrasound probe 14 (hereinafter, referred to as a probe 14) performs transmission/reception of ultrasonic waves, and supplies a reception signal according to a received ultrasonic echo to the diagnostic apparatus body 12.

The probe 14 is a known ultrasound probe which is used in various ultrasound diagnostic apparatuses. The probe 14 has so-called ultrasound transducers (ultrasonic piezoelectric transducers) arranged in a one-dimensional or two-dimensional array which transmit ultrasonic waves toward a subject, receive an ultrasonic echo reflected by the subject, and output an electrical signal (reception signal) according to the received ultrasonic echo.

In the invention, the type of the probe 14 is not particularly limited, and various types, such as a convex type, a linear type, and a sector type, may be used. An external probe or a probe for an ultrasound endoscope, such as a radial scan type, may be used. The probe 14 may have ultrasound transducers for receiving second and higher harmonics of transmitted ultrasonic waves which correspond to harmonic imaging.

In the illustrated example, the probe 14 and the diagnostic apparatus body 12 are connected together by a cable 20. However, the invention is not limited thereto, a transmission circuit 28, a reception circuit 30, a transmission/reception controller 32, and the like described below may be arranged in the probe 14, and the probe 14 and the diagnostic apparatus body 12 may be connected together by wireless communication.

The display 18 is a known display (display device).

In the ultrasound diagnostic apparatus 10, as in various ultrasound diagnostic apparatuses, the display 18 displays an ultrasound image according to a reception signal output from the probe 14, information of the subject, selection means or instruction means for operation by a GUI (Graphical User Interface), a region of interest (hereinafter, referred to as ROI), an elasticity measurement result of a vascular wall described below, and the like.

The operating panel 16 is provided to operate the ultrasound diagnostic apparatus 10.

Though not illustrated, in the ultrasound diagnostic apparatus 10, the operating panel 16 has arranged therein selection means for selecting various modes, such as a B mode and an M mode, a trackball (track pad/touch pad) for moving a cursor, a line, or the like displayed on the display 18, a set button for determining (confirming) selection or operation, a freeze button for switching between motion image display and still image display, changing means for changing the visual field depth of an ultrasound image, gain adjusting means, a zoom button for enlarging an ultrasound image, and the like.

As the modes of the ultrasound diagnostic apparatus 10, in addition to the modes of the normal ultrasound diagnostic apparatus, such as a B mode and an M mode, a VE mode (Vascular Elasticity Mode) for measuring the elastic modulus of the vascular wall is set.

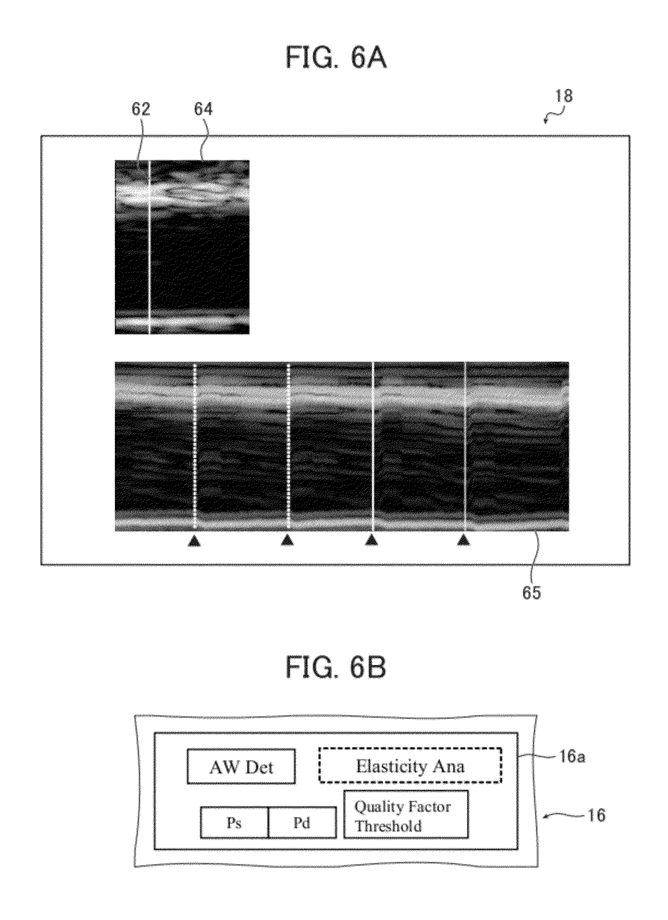

Though not illustrated, the operating panel 16 also has arranged therein a touch panel 16a (see FIG. 6B) which is a display device for operation by GUI.

The diagnostic apparatus body 12 controls the overall operation of the ultrasound diagnostic apparatus 10, and also performs various processes for producing an ultrasound image according to the reception signal output from the probe 14, displaying the ultrasound image on the display 18, and measuring a blood vessel elastic modulus.

The diagnostic apparatus body 12 is constituted using, for example, a computer.

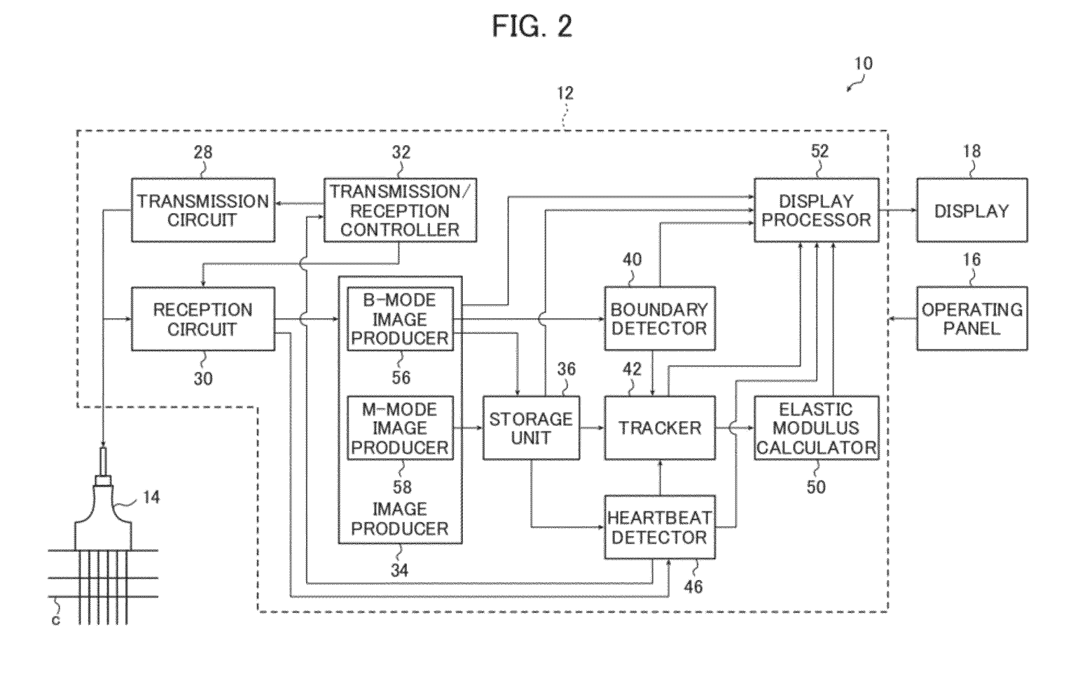

FIG. 2 is a block diagram conceptually illustrating the configuration of the ultrasound diagnostic apparatus 10.

As illustrated in FIG. 2, the diagnostic apparatus body 12 has the transmission circuit 28, the reception circuit 30, the transmission/reception controller 32, an image producer 34, a storage unit 36, a boundary detector 40, a tracker 42, a heartbeat detector 46, an elastic modulus calculator 50, and a display processor 52.

The image producer 34 has a B-mode image producer 56 and an M-mode image producer 58.

The above-mentioned probe 14 is connected to the transmission circuit 28 and the reception circuit 30. The transmission/reception controller 32 is connected to the transmission circuit 28 and the reception circuit 30. The heartbeat detector 46 is connected to the transmission/reception controller 32. The reception circuit 30 is connected to the image producer 34 and the heartbeat detector 46.

The image producer 34 is connected to the display processor 52. The B-mode image producer 56 and the M-mode image producer 58 of the image producer 34 are connected to the storage unit 36. The B-mode image producer 58 is also connected to the boundary detector 40.

The storage unit 36 is connected to the tracker 42, the heartbeat detector 46, and the display processor 52. The heartbeat detector 46 and the boundary detector 40 are connected to the tracker 42 and the display processor 52 together. The tracker 42 is connected to the display processor 52 and the elastic modulus calculator 50, and the elastic modulus calculator 50 is connected to the display processor 52.

The transmission/reception controller 32 controls the actuation of the transmission circuit 28 and the reception circuit 30 to control transmission/reception of ultrasonic waves by the probe 14.

The transmission/reception controller 32 sequentially sets the transmission direction of an ultrasonic beam and the reception direction of an ultrasonic echo of the probe 14 through the transmission circuit 28 and the reception circuit 30. The transmission/reception controller 32 also has a transmission control function of selecting a transmission delay pattern in accordance with the set transmission direction and a reception control function of selecting a reception delay pattern in accordance with the set reception direction.

The transmission delay pattern is the pattern of a delay time which is given to an actuation signal of each ultrasound transducer so as to produce an ultrasonic beam to a desired direction by ultrasonic waves transmitted from a plurality of ultrasound transducers of the probe 14. The reception delay pattern is the pattern of a delay time which is given to a reception signal so as to extract an ultrasonic echo from a desired direction by ultrasonic waves received by a plurality of ultrasound transducers.

A plurality of transmission delay patterns and a plurality of reception delay patterns are stored in an internal memory (not illustrated), and are appropriately selected and used depending on the situation.

The transmission circuit 28 includes a plurality of channels, and produces a plurality of actuation signals which are respectively applied to a plurality of ultrasound transducers of the probe 14. At this time, it is possible to give the delay time to each of a plurality of actuation signals on the basis of the transmission delay pattern selected by the transmission/reception controller 32.

The transmission circuit 28 may adjust the delay amount of each of a plurality of actuation signals such that ultrasonic waves transmitted from a plurality of ultrasound transducers of the probe 14 produce an ultrasonic beam, and may respectively supply the adjusted actuation signals to the ultrasound transducers. Alternatively, the transmission circuit 28 may supply to the probe 14 a plurality of actuation signals made up such that ultrasonic waves transmitted from a plurality of ultrasound transducers at a time cover the entire imaging region of the subject.

Similarly to the transmission circuit 28, the reception circuit 30 includes a plurality of channels. The reception circuit 30 amplifies a plurality of analog signals received through a plurality of ultrasound transducers and converts the amplified analog signals to digital reception signals.

A reception focusing process is performed by giving the delay time to each of a plurality of reception signals on the basis of the reception delay pattern selected by the transmission/reception controller 32 and adding the reception signals. With this reception focusing process, the focus of the ultrasonic echo is narrowed to produce sound ray data (sound ray signal).

As described below in detail, if a ROI 60 is set (the zoom button is depressed), the transmission/reception controller 32 controls the actuation of the transmission circuit 28 and the reception circuit 30 such that ultrasonic wave transmission/reception for obtaining an enlarged B-mode image and an M-mode image of the ROI 60 is performed.

Specifically, in the ultrasound diagnostic apparatus 10 of the invention, the transmission/reception controller 32 controls the actuation of the transmission circuit 28 and the reception circuit 30 such that ultrasonic wave transmission/reception for obtaining a whole image as a B-mode image of a predetermined size including the ROI 60 is incorporated at a predetermined timing when ultrasonic wave transmission/reception for producing the ultrasound image of the ROI 60 is performed.

The B-mode image of the ROI 60 and the whole image produced by the image producer 34 are stored in the storage unit 36 in association with each other.

This will be described below in detail.

The sound ray data produced by the reception circuit 30 is supplied to the image producer 34. The M-mode sound ray data produced by the reception circuit 30 is also supplied to the heartbeat detector 46.

The image producer 34 performs a preprocess, such as Log (logarithmic) compression or gain adjustment, on the supplied sound ray data to produce image data of the ultrasound image, converts (raster-converts) the image data to image data based on a normal television signal scan system, performs a necessary image process, such as a gradation process, on the image data and outputs the image data to the display processor 52.

The image producer 34 has a B-mode image producer 56 which produces a B-mode image, and an M-mode image producer 58 which produces an M-mode image. The B-mode image and the M-mode image may be produced by a known method.

The display processor 52 produces display data for display on the display 18 in accordance with image data of the ultrasound image supplied from the image producer 34, image data of the ultrasound image read from the storage unit 36, operation (input instruction) on the operating panel 16, the measurement result (analysis result) of a vascular wall elastic modulus described below, and the like, and displays the display data on the display 18.

In the ultrasound diagnostic apparatus 10 of the illustrated example, the storage unit 36, the boundary detector 40, the tracker 42, the heartbeat detector 46, and the elastic modulus calculator 50 of the diagnostic apparatus body 12 are primarily used in the VE mode in which the elastic modulus of the vascular wall is measured.

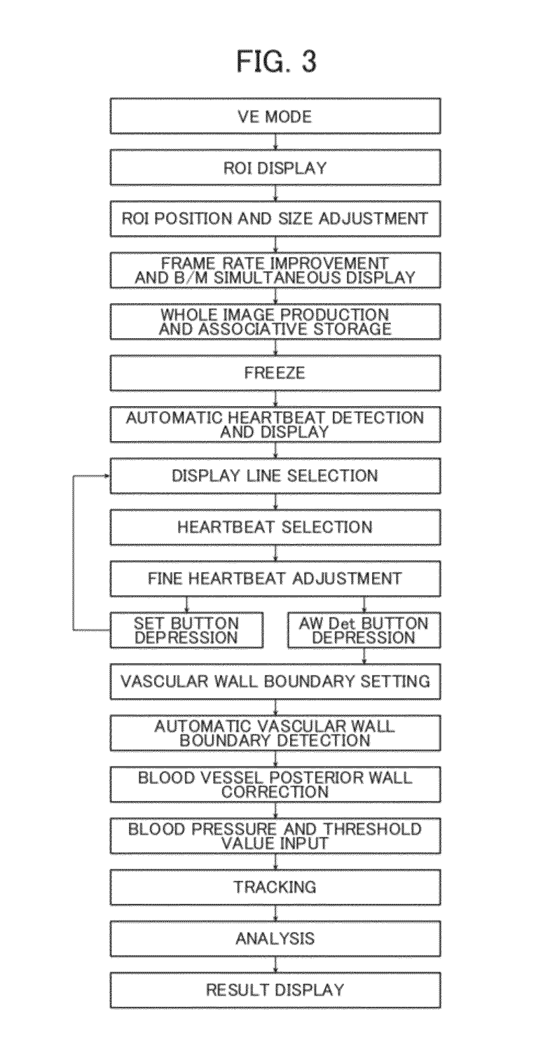

Hereinafter, the ultrasound diagnostic apparatus of the invention will be described in detail by describing the action of the ultrasound diagnostic apparatus 10 in the VE mode with reference to a flowchart of FIG. 3 and FIGS. 5 to 13, and the respective units, such as the storage unit 36 and the elastic modulus calculator 50, will also be described.

In the following description, with regard to the display of the display 18, the display processor 52 performs necessary process, such as line formation, even though not particularly described.

If an ultrasound diagnosis by the ultrasound diagnostic apparatus 10 starts, under the control of the transmission/reception controller 32, the transmission circuit 28 causes the ultrasound transducer of the probe 14 to transmit ultrasonic waves, and the reception circuit 30 processes the reception signal output from the probe 14 to produce sound ray data and outputs the sound ray data to the image producer 34.

As an example, the B mode is selected, as conceptually illustrated in FIG. 4, a carotid artery c of the subject is used as a measurement target, and the probe 14 is brought into contact with a neck n. In this case, a B-mode image produced by the image producer 34 (B-mode image producer 56) is processed by the display processor 52 and displayed on the display 18.

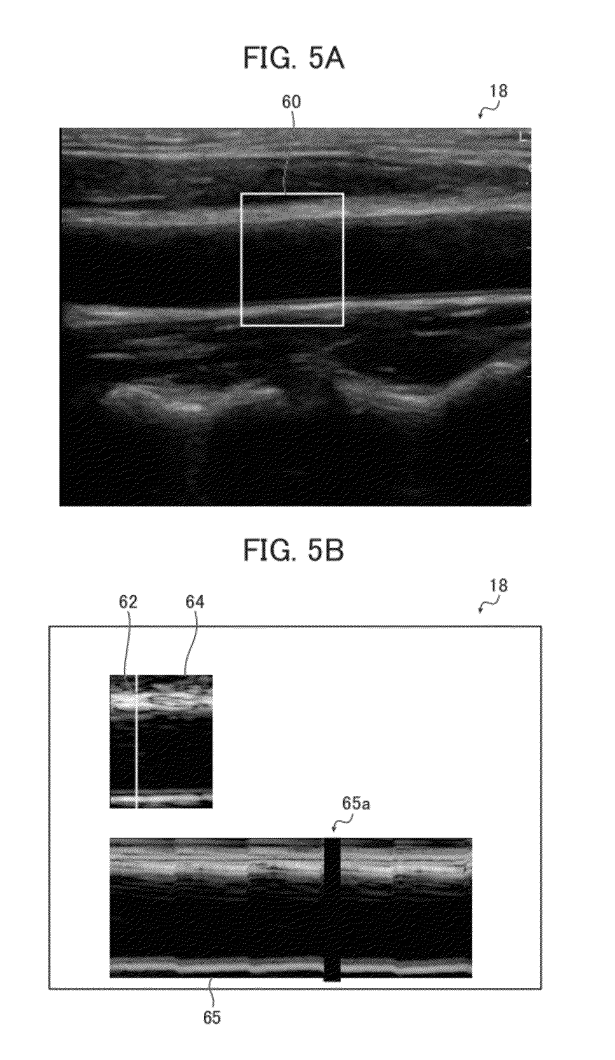

If the intended carotid artery c can be appropriately observed, and the VE mode is selected by mode selection means of the operating panel 16 (in the following description, “of the operating panel 16” is omitted), as conceptually illustrated in FIG. 5A, the display processor 52 displays the ROI 60 representing the region of interest in the B-mode image.

In this state, the position of the ROI 60 in the B-mode image can be moved by operation of the trackball. If the set button is depressed, the position of the ROI 60 is fixed, and the size of the ROI 60 can be changed by operation of the trackball.

Each time the set button is depressed, the position change of the ROI 60 and the size adjustment of the ROI 60 can be alternately carried out.

If the zoom button is depressed in this state, the adjustment of the position or the size of the ROI 60 ends, and the ROI 60 is set.

In response to this situation, the transmission/reception controller 32 increases the frame rate to be higher than before the instruction to set the ROI 60 (for example, to be equal to or higher than 200 Hz, or at least five times higher than before the ROI setting instruction) and controls the actuation of the transmission circuit 28 and the reception circuit 30, that is, the ultrasound transducers of the probe 14 such that ultrasonic wave transmission/reception (ROI transmission/reception) for obtaining the enlarged image of the B-mode image of the ROI 60 and the M-mode image of the ROI 60 is performed.

With the setting of the ROI 60, the B-mode image producer 56 produces the enlarged image of the B-mode image of the ROI 60 in response to the reception signal supplied from the reception circuit 30, and the M-mode image producer 58 starts to produce the M-mode image of the ROI 60. Accordingly, as illustrated in FIG. 5B, a B-mode image 64 (ROI image) where the portion of the ROI 60 is enlarged (zoomed in) and an M-mode image 65 are displayed simultaneously. In the illustrated example, the reception circuit 30 supplies the produced M-mode sound ray data to the heartbeat detector 46.

The simultaneous display (dual mode display) of the B-mode image 64 and the M-mode image 65 may be performed in the same manner as so-called B/M-mode display in the known ultrasound diagnostic apparatus.

In FIG. 5B, the upper side is the B-mode image 64, and the lower side is the M-mode image 65.

In the B-mode image 64, the horizontal direction of the drawing is the azimuth direction (the arrangement direction of the ultrasound transducers (in the two-dimensional arrangement, the longitudinal direction)), and the vertical direction is the depth direction (the transmission/reception direction of ultrasonic waves). The upper side in the depth direction is the side on which the depth is shallow (the probe 14 side).

A selection line 62 which extends in the depth direction to select the display position of the M-mode image (the display line of the M-mode image) in the azimuth direction in the B-mode image is displayed in the B-mode image. The selection line 62 is movable in the azimuth direction (left-right direction) by the trackball.

In the M-mode image 65, the horizontal direction is the direction of the time axis. The time flows from left to right, and the left side of a gap 65a becomes a current frame (that is, the right side of the gap 65a is a previous frame). Similarly to the B-mode image 64, the vertical direction is the depth direction. The upper side in the depth direction is the side on which the depth is shallow.

In FIG. 5B, the M-mode image 65 displayed on the display 18 is the M-mode image 65 at the position of the selection line 62 set in advance.

The M-mode image producer 58 produces an M-mode image at a predetermined position (a predetermined position set in advance or a selected position) in the azimuth direction or a selected position in the azimuth direction as well as over the entire region of the B-mode image 64 in the azimuth direction.

In the ultrasound diagnostic apparatus 10 of the invention, after the ROI 60 is set (that is, after the zoom button is depressed), when performing ultrasonic wave transmission/reception for obtaining the B-mode image 64 and the M-mode image 65, the transmission/reception controller 32 controls the actuation of the transmission circuit 28 and the reception circuit 30 such that ultrasonic wave transmission/reception (whole image transmission/reception) for obtaining the whole image, which is an ultrasound image of a predetermined size including the region of the ROI 60, is performed at a predetermined timing.

In other words, the whole image transmission/reception for obtaining the whole image is incorporated into the ultrasonic wave transmission/reception (ROI transmission/reception) for obtaining the enlarged B-mode image and the M-mode image of the ROI 60 at a predetermined timing.

The B-mode image producer 56 produces the B-mode image of the whole image in accordance with the whole image transmission/reception.

It is preferable that the B-mode image producer 56 acquires and stores information of the set ROI 60 when the ROI 60 is set, and incorporates the set ROI 60 to produce the whole image. That is, it is preferable to reproduce the set ROI 60 in the whole image. The information of the set ROI 60 may be acquired from, for example, the display processor 52 or the transmission/reception controller 32.

The whole image transmission/reception may be performed in the same manner as the B-mode image before the ROI 60 is set. Accordingly, when the whole image transmission/reception is performed, the frame rate is the same as when only the initial B-mode image is displayed.

The whole image may be an image of a size greater than the B-mode image 64 including the region of the ROI 60. Meanwhile, it is preferable that the whole image is an image of the same size as a B-mode image when the ROI 60 is set (when the zoom button is depressed) such that the position of the ROI 60, that is, the position of the blood vessel whose blood vessel elastic modulus has been measured can be easily found later.

Although a single image (single frame) may be basically formed as the whole image, if necessary, a plurality of whole images set appropriately may be formed.

The timing of the whole image transmission/reception (whole image acquisition timing) is not particularly limited, and any timing from when the ROI 60 is set (when the zoom button is depressed) until the freeze button described below is depressed may be set.

As an example, the period from when the ROI 60 is set until the freeze button is depressed is not predicted. Accordingly, after the ROI 60 is set, the whole image transmission/reception may be performed as soon as possible.

In many cases, an image when the freeze button is depressed is stored or analyzed. Accordingly, at the moment the freeze button is depressed, the final ROI transmission/reception may be performed, and the whole image transmission/reception may be subsequently performed.

At whichever timing the whole image transmission/reception is performed after the ROI 60 is set, it is preferable that the whole image transmission/reception is performed at the timing at which the following condition is satisfied.

As described above, the whole image transmission/reception has a low frame rate compared to the transmission/reception for obtaining the B-mode image or the M-mode image of the ROI 60. Accordingly, if the whole image transmission/reception is performed when the motion of the blood vessel is quick, aliasing may occur due to a decrease in the frame rate, causing degradation in image quality of the whole image.

In order to prevent the occurrence of aliasing or the like, it is preferable that the whole image transmission/reception is performed outside the time for which the moving velocity of the vascular wall is high, for example, time from the heart diastole to the heart systole. That is, it is preferable that the timing at which the moving velocity of the vascular wall is low is predicted, and the whole image transmission/reception is performed at the timing at which the moving velocity of the vascular wall is low.

In response to this situation, in the illustrated example, as described above, the reception circuit 30 supplies M-mode sound ray data to the heartbeat detector 46.

The heartbeat detector 46 detects the moving velocity of the vascular wall from the supplied M-mode sound ray data, predicts the time at which the moving velocity of the vascular wall is highest, and supplies the information to the transmission/reception controller 32. The transmission/reception controller 32 controls the actuation of the transmission circuit 28 and the reception circuit 30 such that the whole image transmission/reception is performed (the whole image is acquired) when a predetermined time has elapsed after the moving velocity of the vascular wall is highest.

The predetermined time after the moving velocity of the vascular wall is not particularly limited and may be appropriately set. Normally, one human heartbeat takes about one second. Accordingly, the whole image transmission/reception is performed when about 0.2 to 0.5 seconds have elapsed after the moving velocity of the blood vessel is highest, such that the whole image transmission/reception can be performed at the timing at which the moving velocity of the vascular wall is low. The predetermined time may be set by the operator.

Alternatively, the heartbeat detector 46 may detect the moving velocity of the vascular wall in a similar manner and also may predict the length of one heartbeat from the moving velocity of the vascular wall, and the whole image transmission/reception may be performed when about ¼ of one heartbeat has elapsed after the moving velocity of the vascular wall is highest.

The heartbeats may be detected as described below, the length of one heartbeat may be predicted from the moving velocity of the vascular wall in a similar manner, and the whole image transmission/reception may be performed about the center of the heartbeat.

In the above example, the heartbeat detector 46 predicts the time at which the moving velocity of the vascular wall is highest or the length of the heartbeat using M-mode sound ray data. However, the invention is not limited thereto, heartbeat detection or the like may be performed using an electrocardiograph (electrocardiogram), and the timing of the whole image transmission/reception may be set in a similar manner.

The whole transmission/reception for forming a single whole image is not limited to continuous transmission/reception for one frame.

For example, the whole image is divided into, for example, three images in the horizontal direction on the display 18. First, whole image transmission/reception for obtaining a left ⅓ whole image is performed. Next, ROI transmission/reception for a predetermined number of frames is performed. Next, whole image transmission/reception for obtaining a central ⅓ whole image is performed. Next, ROI transmission/reception for a predetermined number of frames is performed. Next, whole image transmission/reception for obtaining a right ⅓ whole image is performed. In this way, a single whole image may be formed.

The B-mode image (B-mode image data) of the ROI 60 produced by the B-mode image producer 56 and the M-mode image (M-mode image data) produced by the M-mode image producer 58 are stored in the storage unit 36 together.

The temporal amount of an image which is stored in the storage unit 36 is not particularly limited, while a duration including two or more common heartbeats is preferred. Accordingly, it is preferable that the storage unit 36 stores the latest B-mode image and M-mode image of the ROI 60 that are each three seconds or longer in duration.

In the invention, the whole image is also stored in the storage unit 36 in association with the B-mode image of the ROI 60 and, optionally, the M-mode image as well. The information of the subject or the measurement information such as the date is also associated with these images.

As described above, the selection line 62 can be moved in the azimuth direction by the trackball.

The position of the selection line 62 and the M-mode image are moved together. That is, if the selection line 62 is moved in the left-right direction by the trackball, the display processor 52 displays the M-mode image of the position of the selection line 62 on the display 18.

The operator depresses the freeze button if it is determined that an appropriate image is obtained.

If the freeze button is depressed, the display processor 52 reads necessary image data from the storage unit 36, and as illustrated in FIG. 6A, the display processor 52 rearranges the M-mode image 65 of the position of the selection line 62 such that the time at which the freeze button is depressed is on the rightmost side (latest position) and displays the M-mode image 65 with the still image of the B-mode image 64 on the display 18. Simultaneously, the selection line 62 becomes a broken line and is not movable (inactive state).

As illustrated in FIG. 6B, an “AW Det” button for instructing to set the boundary of the vascular wall described below, an “Elasticity Ana” button for instructing to start the analysis of the vascular wall elastic modulus, a “Ps” button and a “Pd” button for inputting the blood pressure of the subject, and a “Quality Factor Threshold” button for inputting a reliability threshold value are displayed on the touch panel 16a of the operating panel 16. At this time, the “Elasticity Ana” button is not selectable.

If the freeze button is depressed, the heartbeat detector 46 detects the heartbeats (automatically detects the heartbeats) for all the M-mode images stored in the storage unit 36. The detection result of the heartbeats is sent to the storage unit 36, and added to the corresponding M-mode image as information.

The detection result of the heartbeats is also sent to the display processor 52, and the detection result of the heartbeats is displayed in the M-mode image 65 which is currently displayed.

The method of detecting the heartbeats is not particularly limited. As an example, an M-mode image may be analyzed, and the heartbeats may be detected using the moving velocity (the time at which the velocity starts to increase) in the depth direction of a white line (bright line) extending in the horizontal direction, the pulsation of the motion in the depth direction of the white line, or the like. Alternatively, an electrocardiograph (electrocardiogram) may be used to detect the heartbeats.

As illustrated in FIG. 6A, the display processor 52 displays the detection result of the heartbeats in the M-mode image 65 by a triangular mark and a line. In the illustrated example, the time at which the latest heartbeat starts is indicated by a solid line, the time at which the same heartbeat ends is indicated by a thin line, and the position related to other heartbeats is indicated by a broken line. The lines may be distinguished by changing the line color instead of or in addition to the line type.

When there is the heartbeat which is not detected, the heartbeat is displayed at an appropriate position in accordance with the interval of heartbeats prior to and subsequent to the heartbeat in question, or the like.

The B-mode image 64 when the freeze button is depressed is a B-mode image at the time when the latest heartbeat starts, with the time being indicated in the M-mode image 65 by a solid line.

If the lines of the heartbeats are displayed in the M-mode image 65, the selection line 62 in the B-mode image becomes a solid line and is movable in the left-right direction by the trackball. That is, the selection line 62 is in the active state. Whether or not the line is active may be distinguished by changing the line color instead of or in addition to the line type in a similar manner to the above.

In this state, if the selection line 62 is moved in the left-right direction by the trackball, the display processor 52 reads an M-mode image corresponding to the position of the selection line 62 from the storage unit 36, and displays the image on the display 18 along with the detection result of the heartbeats. That is, the selection line 62 is moved by the trackball even after freeze, thereby selecting the display position (display line) of the M-mode image 65 in the B-mode image 64 over the entire region in the azimuth direction in the B-mode image 64.

Therefore, according to this example, the M-mode image 65 of an arbitrary position in the azimuth direction of the set ROI 60 is displayed, such that the M-mode image 65 and an image corresponding to each heartbeat in the M-mode image can be observed and confirmed.

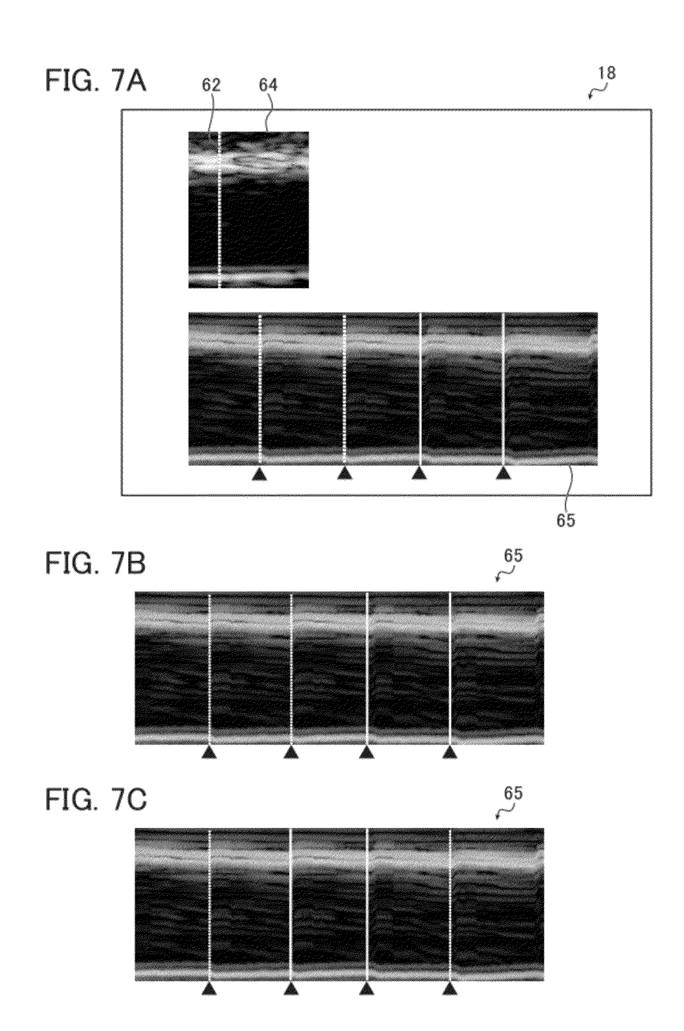

If the set button is depressed in a state where the selection line 62 of the B-mode image 64 is movable, it is determined that the display position (display line) of the M-mode image is selected. As illustrated in FIG. 7A, the selection line 62 of the B-mode image 64 becomes a broken line, such that the movement by the trackball is impossible. Simultaneously, lines indicating the latest heartbeat become a solid line in the M-mode image 65.

If the lines indicating the latest heartbeat become a solid line in the M-mode image 65, the heartbeat is selectable by the trackball.

As an example, when the set button is depressed, as illustrated in FIGS. 7A and 7B, the lines indicating the latest heartbeat become a solid line, and the heartbeat is selected. In this state, for example, if the trackball rotates left, as illustrated in FIG. 7C, a line corresponding to the end of the latest heartbeat becomes a broken line, lines corresponding to the second latest heartbeat become a solid line, and the heartbeat is selected. If the trackball further rotates left, lines corresponding to the second latest heartbeat become a broken line, lines corresponding to the third latest heartbeat become a solid line, and the heartbeat is selected.

If the trackball rotates right, similarly, lines corresponding to later heartbeats are sequentially selected.

In response to the selection of the heartbeat, the display processor 52 reads from the storage unit 36, the B-mode image at the start position of the selected heartbeat, that is, the B-mode image which is captured at the time (time phase) corresponding to the start position of the selected heartbeat, and changes the B-mode image 64 displayed on the display 18 to this image.

If the set button is depressed in a state where the heartbeats are selectable, it is determined that the selection of the heartbeats ends, the selected heartbeat is confirmed, and fine adjustment of the selected heartbeat can be performed.

If a heartbeat in the M-mode image 65 displayed on the display 18 is selected and confirmed, the same heartbeat is selected in all the M-mode images (that is, the M-mode images over the entire region in the azimuth direction of the B-mode image 64) stored in the storage unit 36.

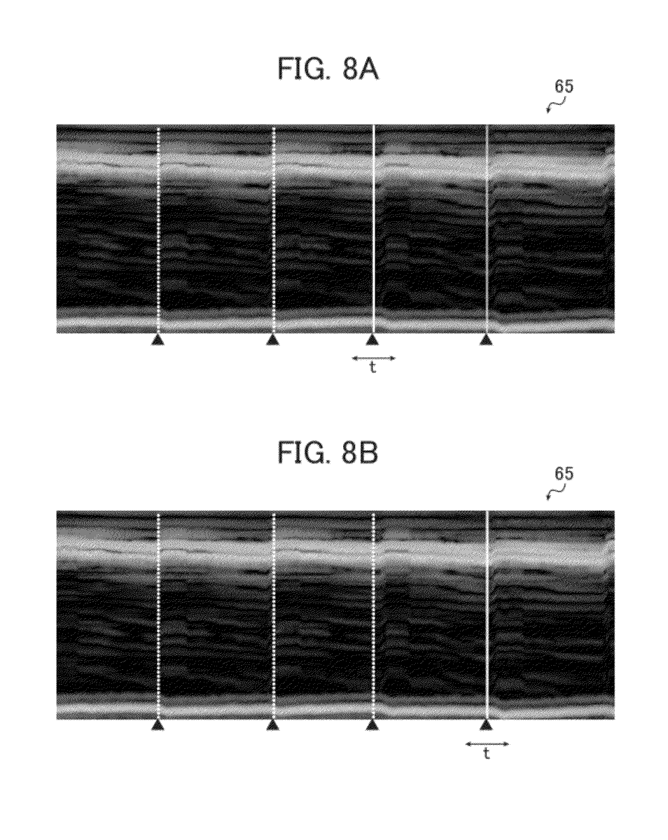

As an example, if it is determined that the latest heartbeat is selected and the set button is depressed, as illustrated in FIG. 8A, first, a line corresponding to the end of the selected heartbeat becomes a thin line, and the position (time) of a line corresponding to the start of the selected heartbeat is movable in the left-right direction (time direction) by the trackball as indicated by an arrow t, such that fine adjustment of the start position of the heartbeat can be performed.

If the set button is depressed after the start position of the heartbeat is adjusted by the trackball as required, as illustrated in FIG. 8B, a line corresponding to the end of the selected heartbeat becomes a normal solid line, and a line corresponding to the start of the selected heartbeat becomes a thin line. Accordingly, the position of the line corresponding to the end of the selected heartbeat is movable in the left-right direction by the trackball as indicated by the arrow t, such that fine adjustment of the end position of the heartbeat can be performed.

Although the result of fine adjustment of the heartbeat may be reflected only in the M-mode image 65 subjected to fine adjustment, it is preferable that the result is also reflected in all the M-mode images stored in the storage unit 36.

When the start position of the heartbeat is adjusted, the display processor 52 reads the B-mode image at the adjusted heartbeat start position from the storage unit 36, and the B-mode image 64 displayed on the display 18 is changed to this image.

The results of heartbeat selection and possible fine adjustment are also supplied to the tracker 42.

If the set button is depressed in a state where the position corresponding to the end of the selected heartbeat is adjustable, the state where the selection line 62 of the B-mode image 64 illustrated in FIG. 6A is movable, that is, the state where the display line of the M-mode image 65 is selectable in the B-mode image 64 is returned.

That is, in the ultrasound diagnostic apparatus 10 of the illustrated example, the processes “display line selection”→“heartbeat selection”→“heartbeat fine adjustment” can be repeatedly performed. In other words, the processes “display line selection”→“heartbeat selection”→“heartbeat fine adjustment” may be performed in a looped manner.

Accordingly, it becomes possible to more suitably select the heartbeat most appropriate for analysis to measure the vascular wall elasticity described below from all the stored M-mode images.

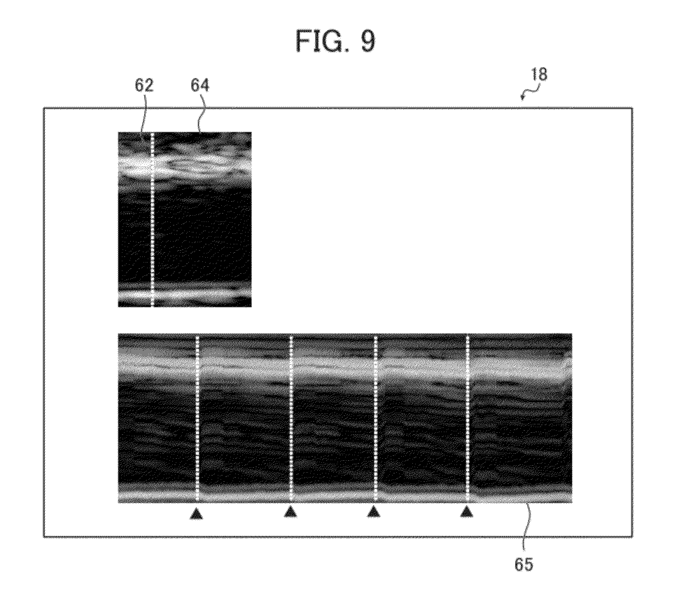

If the “AW Det” button of the touch panel, not the set button, is depressed in a state where the position corresponding to the end of the selected heartbeat is adjustable, as illustrated in FIG. 9, the selection line 62 of the B-mode image 64 and the lines representing the heartbeats in the M-mode image 65 all become a broken line and are inoperable, and a vascular wall detection mode is reached.

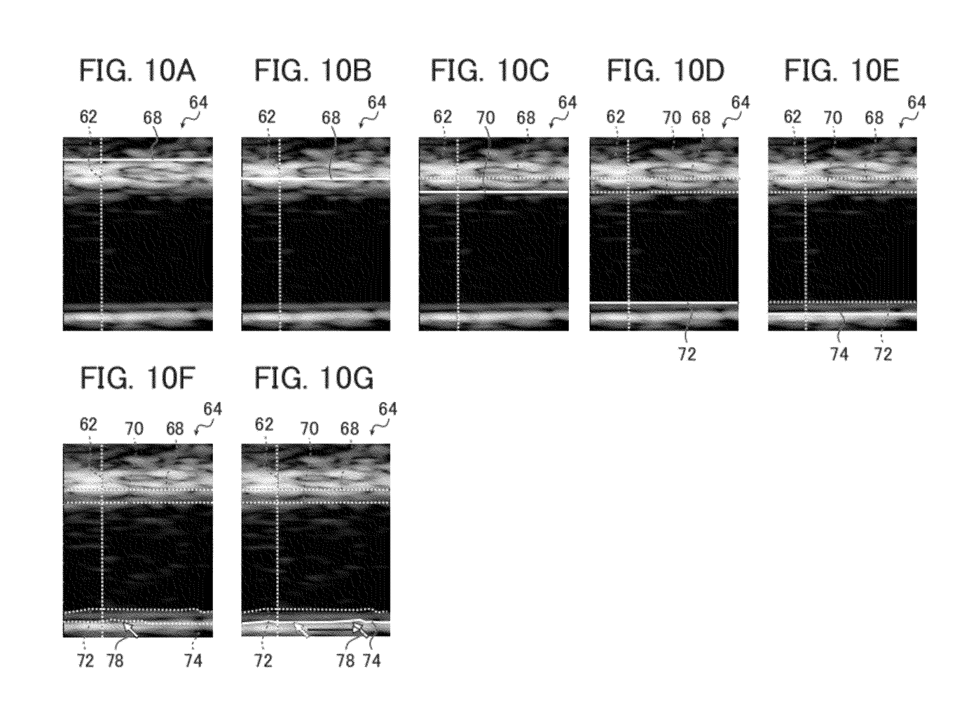

If the vascular wall detection mode is reached, first, as illustrated in FIG. 10A, a line 68 corresponding to the adventitia-media boundary of the blood vessel anterior wall is displayed in the B-mode image 64.

The line 68 is parallel-movable in the up-down direction (depth direction) by the trackball. As illustrated in FIG. 10B, after the line 68 is moved to the position of the adventitia-media boundary of the blood vessel anterior wall by the trackball, the set button is depressed.

If the set button is depressed, as illustrated in FIG. 10C, the line 68 corresponding to the adventitia-media boundary of the blood vessel anterior wall becomes a broken line and is confirmed in the B-mode image 64, and a line 70 corresponding to the intima-lumen boundary of the blood vessel anterior wall is displayed.

Similarly, the line 70 is movable in the up-down direction by the trackball, and after the line 70 is moved to the position of the intima-lumen boundary of the blood vessel anterior wall, the set button is depressed.

If the set button is depressed in a state where the line 70 is movable, as illustrated in FIG. 10D, the line 70 corresponding to the intima-lumen boundary of the blood vessel anterior wall becomes a broken line and is confirmed in the B-mode image 64, and a line 72 corresponding to the intima-lumen boundary of the blood vessel posterior wall is displayed. Similarly, after the line 72 is moved to the position of the intima-lumen boundary of the blood vessel posterior wall by the trackball, the set button is depressed.

If the set button is depressed in a state where the line 72 is movable, as illustrated in FIG. 10E, the line 72 corresponding to the intima-lumen boundary of the blood vessel posterior wall becomes a broken line and is confirmed in the B-mode image 64, and a line 74 corresponding to the adventitia-media boundary of the blood vessel posterior wall is displayed. Similarly, after the line 74 is moved to the position of the adventitia-media boundary of the blood vessel posterior wall by the trackball, the set button is depressed.

The information of each boundary of the vascular wall is supplied to the boundary detector 40.

If the set button is depressed in a state where the line 74 is movable, the setting of the lines corresponding to all the boundaries ends, and the boundary detector 40 automatically detects the intima-lumen boundary and the adventitia-media boundary of the posterior wall using the set line 72 of the intima-lumen boundary and the set line 74 of the adventitia-media boundary. The result of the automatic detection of both boundaries is sent to the display processor 52 and the tracker 42, and as illustrated in FIG. 10F, the detection result is displayed.

The method of automatically detecting these boundaries is not particularly limited, and various methods may be used. As an example, a method is used in which a B-mode image is analyzed, continuous high-luminance portions at the positions of the line 72 and the line 74 are tracked to detect the intima-lumen boundary and the adventitia-media boundary.

If the automatic detection of the intima-lumen boundary and the adventitia-media boundary of the blood vessel posterior wall by the boundary detector 40 ends, as illustrated in FIG. 10F, a cursor 78 is displayed in the B-mode image 64 (the cursor 78 is not displayed until the automatic detection of the blood vessel posterior wall ends).

The cursor 78 is movable by the trackball. If the cursor 78 is moved to the line representing the automatically detected intima-lumen boundary or adventitia-media boundary, and the set button is depressed, the line closer to the cursor 78 becomes a solid line. The line which has become a solid line is correctable.

For example, as illustrated in FIG. 10G, it is assumed that the line 74 representing the adventitia-media boundary is selected and becomes a solid line. If the cursor 78 is moved along the line 74 by the trackball, and the set button is depressed again, the line 74 of the region tracked by the cursor is detected again by the boundary detector 40 and rewritten, and the result is sent to the tracker 42.

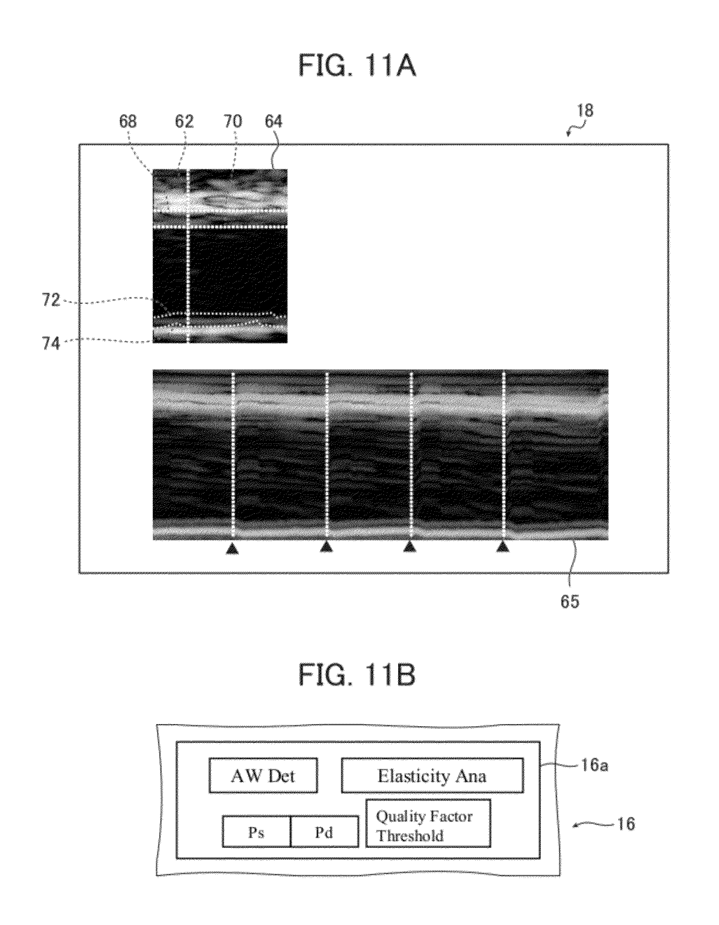

If the automatic detection of the intima-lumen boundary and the adventitia-media boundary of the posterior wall ends, and if necessary, the blood vessel posterior wall is corrected, as illustrated in FIG. 11A, all lines become a broken line, and as illustrated in FIG. 11B, the “Elasticity Ana” button of the touch panel 16a is selectable.

After the “Elasticity Ana” button is selectable, the blood pressure in the heart systole of the subject is input by the “Ps” button, the blood pressure in the heart end diastole of the subject is input using the “Pd” button, and the reliability threshold value is input using the “Quality Factor Threshold” button. These numerical values may be input by a known method.

The input of the blood pressure of the subject and the reliability threshold value is not limited to the input after the detection of the vascular wall boundaries has ended. The input may be performed at any timing before analysis described below starts (before the “Elasticity Ana” button described below is depressed).

In the ultrasound diagnostic apparatus 10, it is usual that before a diagnosis is performed, the subject information is acquired or input. Accordingly, when the subject information includes the information of the blood pressure, the information of the blood pressure may be used.

If the blood pressure of the subject and the reliability threshold value are input, and the “Elasticity Ana” button is depressed, image analysis starts, and the elastic modulus of the vascular wall is calculated.

If the “Elasticity Ana” button is depressed, first, the tracker 42 tracks the motions of the blood vessel anterior wall (adventitia-media boundary and intima-lumen boundary) and the blood vessel posterior wall (intima-lumen boundary and adventitia-media boundary) in the selected heartbeat in the M-mode image 65. That is, the blood vessel anterior wall and posterior wall are tracked.

The tracking of the vascular wall in the M-mode image 65 is performed with the adventitia-media boundary of the blood vessel anterior wall, the intima-lumen boundary of the blood vessel anterior wall, the intima-lumen boundary of the blood vessel posterior wall, and the adventitia-media boundary of the blood vessel posterior wall previously detected (with the lines set) in the B-mode image 64 as a positional starting point (a starting point in the depth direction).

In regard to the tracking of the vascular wall in the M-mode image 65, a temporal starting point (a starting point on the time axis of the M-mode image) is the time phase of the B-mode image 64, that is, the time at which the B-mode image 64 is captured. That is, in the illustrated example, the start position of the heartbeat which is selected and, if necessary, adjusted in position becomes the temporal starting point for the tracking of the vascular wall.

In the ultrasound diagnostic apparatus 10, as a preferred form, not only the detected (set) boundaries of the vascular wall but also one or more measurement points in the depth direction may be set in the blood vessel posterior wall. In this way, when one or more measurement points are set in the blood vessel posterior wall, the tracking of the vascular wall is performed at each measurement point.

The measurement point in the vascular wall may be set in advance, may be automatically set on the basis of a specific algorithm, or may be set by the operator of the ultrasound diagnostic apparatus 10 while viewing the image. These may be used in combination.

The method of tracking the vascular wall in the M-mode image 65 is not particularly limited, and there are a method which uses continuity of images (luminance) from the starting point of the tracking, a pattern matching method, a zero crossing method, a tissue Doppler method, phase difference tracking, and the like. Of these, any method may be used.

The tracking result of the vascular wall in the M-mode image by the tracker 42 is supplied to the elastic modulus calculator 50 and the display processor 52.

The elastic modulus calculator 50 first produces the change waveform of the thickness of the vascular wall (intima-media) and the change waveform of the blood vessel diameter (inner diameter) from the tracking result of the vascular wall. As described above, when one or more measurement points are set in the vascular wall, the change waveform of the vascular wall is produced between the measurement points.

The change waveform of the thickness of the vascular wall and the change waveform of the blood vessel diameter are sent to the display processor 52.

The elastic modulus calculator 50 calculates strain in the radial direction of the blood vessel using Equation (1).

εᵢ=Δhᵢ/h_{di} (1)

In Equation (1), εᵢ denotes strain in the radial direction of the blood vessel between the measurement points, Δhᵢ denotes the maximum value of a change in thickness of the vascular wall between the measurement points in the heart systole in which the vascular wall is smallest in thickness in one heartbeat, and h_{di} denotes the thickness between the measurement points in the heart end diastole in which the vascular wall is largest in thickness.

The elastic modulus calculator 50 calculates an elastic modulus E_{θi} in the circumferential direction of the vascular wall by Equation (2) using the maximum value and the minimum value of the blood pressure input in advance.

E_{θi}=[ 1/2]*[1+(r_{d}/h_{d})]*[Δp/(Δhᵢ/h_{di})] (2)

An elastic modulus Eᵣᵢ in the radial direction of the vascular wall may be calculated by Equation (3).

Eᵣᵢ=Δp/(Δhᵢ/h_{di}) (3)

In Equations (2) and (3), Δhᵢ and h_{di} are the same as described above, Δp denotes a blood pressure difference between the heart systole and the heart end diastole, r_{d} denotes the radius of the vascular lumen in the heart end diastole, and h_{d} denotes the thickness of the vascular wall in the heart end diastole.

After the elastic modulus is calculated, the elastic modulus calculator 50 calculates reliability of the elastic modulus.

The method of calculating reliability of the elastic modulus is not particularly limited, and various known methods may be used. As an example, there is a method in which the waveforms of changes in the blood vessel diameter by the heartbeats of many people, such as 1000 persons are prepared, the model waveform of the change in the blood vessel diameter is created from many waveforms, and reliability of the calculated elastic modulus is calculated using the amount of a shift from the model waveform.

As described above, if a heartbeat is selected and confirmed in the M-mode image displayed on the display 18, the same heartbeat is selected in all the M-mode images stored in the storage unit 36.

Accordingly, the processes, such as the tracking of the vascular wall, the production of the change waveforms of the thickness of the vascular wall and the blood vessel diameter, the calculation of strain of the vascular wall, and the calculation of the elastic modulus of the vascular wall and reliability of the elastic modulus, are performed in the selected heartbeat for not only the M-mode image 65 displayed on the display 18 but also all the M-mode images stored in the storage unit 36. That is, the processes, such as calculation of the elastic modulus of the vascular wall, in the selected heartbeat are performed over the entire region in the azimuth direction of the B-mode image 64 displayed on the display 18 using the corresponding M-mode images.

These results are added to the M-mode images stored in the storage unit 36 as information.

After the calculation over the entire region in the azimuth direction ends, the elastic modulus calculator 50 calculates the average value (E_{θave}) of the elastic modulus of the vascular wall, the average value (Strₐᵥₑ) of strain of the vascular wall, and the average value (QFₐᵥₑ) of reliability of the elastic modulus, and the calculation results are added to the images stored in the storage unit 36 as information.

If the calculation ends, the result is displayed on the display 18.

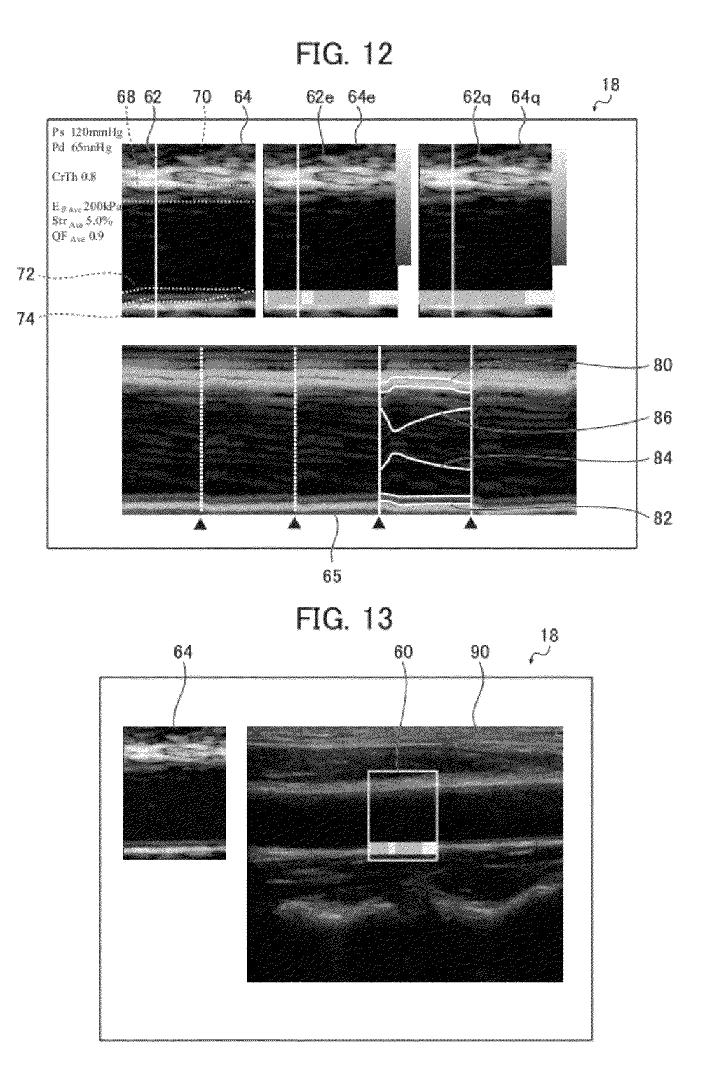

FIG. 12 illustrates an example. In the illustrated example, on the right side of the displayed B-mode image 64, the elastic modulus of the blood vessel posterior wall represented in the B-mode image 64 is displayed by a B-mode image 64e. On the right side of the B-mode image 64e which displays the elastic modulus of the blood vessel posterior wall, reliability of the elastic modulus of the vascular wall is displayed by a B-mode image 64q in a similar manner.

On the left side of the B-mode image 64, the average value (E_{θave}) of the elastic modulus of the vascular wall, the average value (Strₐᵥₑ) of strain of the vascular wall, and the average value (QFₐᵥₑ) of reliability of the elastic modulus are respectively displayed.

The elastic modulus of the vascular wall is displayed in a strip shape in the B-mode image 64e to overlap the blood vessel posterior wall automatically detected (and corrected as necessary) in the B-mode image 64. On an upper right side of the B-mode image 64e, the index of the elastic modulus is displayed. In the illustrated example, the higher the image density, the higher the elastic modulus.

That is, in the B-mode image 64e, the density of the strip overlapping the blood vessel posterior wall represents the elastic modulus of the vascular wall at the corresponding position of the blood vessel.

Similarly, reliability of the elastic modulus is displayed in a strip shape in the B-mode image 64q to overlap the blood vessel posterior wall automatically detected in the B-mode image 64. On an upper right side of the B-mode image 64q, the index of reliability of the elastic modulus is displayed. In the illustrated example, the higher the image density, the higher reliability of the elastic modulus.

That is, in the B-mode image 64q, the density of the strip overlapping the blood vessel posterior wall represents reliability of the vascular wall elastic modulus at the corresponding position of the blood vessel.

The magnitude of the elastic modulus or reliability of the elastic modulus may be realized by changing the image color instead of or in addition to the image density.

In the ultrasound diagnostic apparatus 10 of the invention, the whole image including the ROI 60 subjected to analysis and blood vessel elasticity measurement is stored.

Accordingly, as shown in FIG. 13, a whole image 90 and the B-mode image 64 (an enlarged B-mode image of the ROI 60) may be displayed on the display 18, and the measurement result of the blood vessel elasticity may similarly be displayed in the ROI 60 of the whole image 90.

If there is a space in the display screen of the display 18, or by reducing the size of the whole image 90 or the like to secure a space in the display screen, the whole image 90 displaying the measurement result of the blood vessel elasticity, the B-mode image 64, and the M-mode image 65 (the M-mode image of the ROI 60) displaying the result may be displayed on the display 18.

The B-mode image 64q representing reliability may also be displayed if there is a space in the display screen or by securing a display space by means of reduction of image size or the like.

The display of the measurement result not including the whole image 90 shown in FIG. 12 or the display of the measurement result including the whole image 90 shown in FIG. 13 may be selected by the operator, or the display may be switched in a toggled manner, using the operating panel 16 or the like.

The images to be displayed including the whole image may be selected by the operator.

In the display of the result shown in FIG. 12 (or FIGS. 12 and 13), the result is automatically omitted at the position in the azimuth direction where reliability of the result is lower than a threshold value input in advance.

With regard to the position where the result is omitted, as represented in a right corner portion of the result display of the elastic modulus in the B-mode image 64e or a right corner portion of the result display of reliability in the B-mode image 64q, the display of the strip is thinned.

In the lower M-mode image 65, a tracking result 80 of the blood vessel anterior wall, a tracking result 82 of the blood vessel posterior wall, a change waveform 84 of the blood vessel diameter, and a change waveform 86 of the thickness of the vascular wall in the M-mode image are displayed in the selected heartbeat.

As described above, when one or more measurement points are set in the vascular wall in the depth direction, the change waveform of the blood vessel thickness may be output between the measurement points.

If the measurement result of the elastic modulus of the vascular wall or the like is displayed on the display 18, the selection line 62 becomes a solid line in the B-mode image 64, and is movable in the azimuth direction by the trackball.

If the selection line 62 is moved in the B-mode image 64, the display processor 52 reads the M-mode image corresponding to the position of the selection line 62 from the storage unit 36 and displays the M-mode image on the display 18. That is, if the selection line 62 is moved by the trackball, the M-mode image 65 is changed to the M-mode image at the position of the selection line 62, and the tracking results 80 and 82 of the blood vessel anterior wall and the blood vessel posterior wall, the change waveform 84 of the blood vessel diameter and the change waveform 86 of the thickness of the vascular wall in the M-mode image are changed to data at the position of the selection line 62 of the B-mode image 64.

Accordingly, it is possible to select the display line for displaying the M-mode image 65 and the analysis result over the entire region in the azimuth direction of the B-mode image.

After the set button is depressed, in the B-mode image 64e and the B-mode image 64q, if a selection line 62e and a selection line 62q are moved by the trackball to select an arbitrary region in the azimuth direction, and thereafter, the set button is depressed again, the selected region is handled in a similar manner to the above-mentioned region where reliability is lower than the threshold value, and data is deleted.

That is, a tester views the result, and when there is a location where the waveform or the like seems to be extraordinary, data can be deleted, thereby making it possible to perform more accurate analysis.

The state after the deletion of data may be returned in a previous state by depressing a Delete button or the like.

As described above, in the ultrasound diagnostic apparatus 10 of the invention, the storage unit 36 stores a B-mode image of the ROI 60 where the blood vessel elasticity is measured and a whole image of a predetermined size including the ROI 60 (for example, a B-mode image of the same size as when the ROI is set) in association with each other.

Accordingly, when the blood vessel elasticity is measured again for the subject who was subjected to the previous measurement of the blood vessel elasticity, a B-mode image and a corresponding whole image which are stored can be retrieved in accordance with an input instruction by the operating panel 16. Then, for example, in a similar manner to FIG. 13, the B-mode image and the corresponding whole image can be displayed on the display 18 together.

Therefore, the tester can easily and accurately find the position of the blood vessel or the like which was measured in the previous measurement of the subject, and can make a new measurement. For this reason, according to the invention, it is possible to do follow-up satisfactorily, thereby making an appropriate diagnosis.

In the ultrasound diagnostic apparatus 10 of the invention, a whole image which is retrieved and displayed in accordance with a new measurement may be a whole image including the measurement result (analysis result) of the blood vessel elasticity illustrated in FIG. 13 or a whole image not including the measurement result.

In the ultrasound diagnostic apparatus 10, the images which are displayed in accordance with an input instruction for retrieval are not limited to the B-mode image of the ROI 60 and the whole image, and various combinations may be used in accordance with images stored in the storage unit 36.

For example, only the whole image may be displayed, the whole image and the B-mode image and the M-mode image of the ROI 60 may be displayed in a similar way as described above, the whole image and the M-mode image may be displayed, or the whole image and the B-mode image 64e representing the result of the blood vessel elasticity may be displayed.

The image (retrieved image) which is displayed along with the whole image may be selected by the operator using the operating panel 16.

The B-mode image of the ROI 60 where the blood vessel elasticity is measured and the associated whole image (and even the M-mode image) may be stored in an external storage device which is connected (connectable) to the ultrasound diagnostic apparatus 10, not in the storage unit 36 embedded in the ultrasound diagnostic apparatus 10, and retrieved.

Although the ultrasound diagnostic apparatus of the invention has been described in detail, the invention is not limited to the foregoing examples, and various modifications or improvements may be of course made without departing from the scope of the invention.

While the above-described examples are examples where the ultrasound diagnostic apparatus of the invention is used in an apparatus capable of measuring the blood vessel elasticity, the invention is not limited thereto. That is, the invention can be used in various ultrasound diagnostic apparatuses in each of which a ROI is set in an ultrasound image and the ROI is enlarged (displayed on an enlarged scale), for example, an ultrasound diagnostic apparatus in which the intima-media thickness (IMT) is measured.

The ultrasound diagnostic apparatus of the invention can be suitably used in medical practice for the diagnosis of arteriosclerosis which causes myocardial infarction, angina pectoris, brain diseases, and the like.

What is claimed is:

1. An ultrasound diagnostic apparatus comprising:
an ultrasound probe which has ultrasound transducers transmitting ultrasonic waves, receiving an ultrasonic echo reflected by a subject, and outputting a reception signal according to the received ultrasonic echo;
an actuation controller for controlling transmission/reception of ultrasonic waves by the ultrasound transducers;
an image producer for producing an ultrasound image from the reception signal output from the ultrasound transducers;
a region-of-interest setter for setting a region of interest in the ultrasound image of a predetermined size produced by the image producer; and
a storage unit for storing the ultrasound image produced by the image producer,
wherein after the region of interest is set by the region-of-interest setter, the actuation controller controls transmission/reception of ultrasonic waves by the ultrasound transducers such that region-of-interest transmission/reception which is transmission/reception of ultrasonic waves corresponding to the region of interest is performed, and whole image transmission/reception for obtaining the ultrasound image of the predetermined size including the set region of interest is incorporated into the region-of-interest transmission/reception at a predetermined timing,
after the region of interest is set by the region-of-interest setter, the image producer produces a region-of-interest image which is obtained by processing a reception signal by the region-of-interest transmission/reception and in which the region of interest is enlarged relative to the ultrasound image of the predetermined size, and the whole image which is the ultrasound image of the predetermined size obtained by processing a reception signal by the whole image transmission/reception, and
the storage unit stores the region-of-interest image and the whole image including the region of the region-of-interest image in association with each other.

2. The ultrasound diagnostic apparatus according to claim 1, further comprising:
a moving velocity detector for detecting a moving velocity of a vascular wall,
wherein the actuation controller predicts a time, at which the moving velocity of the vascular wall is highest, in accordance with a detection result of the moving velocity of the vascular wall by the moving velocity detector, and incorporates the whole image transmission/reception into the region-of-interest transmission/reception when a predetermined period has elapsed from the predicted highest velocity time.

3. The ultrasound diagnostic apparatus according to claim 1, further comprising:
a heartbeat detector for detecting a heartbeat,
wherein the actuation controller incorporates the whole image transmission/reception into the region-of-interest transmission/reception when a predetermined period has elapsed from a start of the heartbeat in accordance with a detection result of the heartbeat by the heartbeat detector.

4. The ultrasound diagnostic apparatus according to claim 1,
wherein the actuation controller controls transmission/reception of ultrasonic waves by the ultrasound transducers such that a frame rate of the region-of-interest transmission/reception is higher than frame rates of ultrasonic wave transmission/reception for obtaining the ultrasound image of the predetermined size where the region of interest is set, and of the whole image transmission/reception.

5. The ultrasound diagnostic apparatus according to claim 1,
wherein after the region of interest is set by the region-of-interest setter, the image producer produces a B-mode image of the region of interest as the region-of-interest image and an M-mode image of the region of interest from a reception signal by the region-of-interest transmission/reception, and produces a B-mode image of a predetermined size as the whole image from a reception signal by the whole image transmission/reception.

6. The ultrasound diagnostic apparatus according to claim 5,
wherein, after the region of interest is set by the region-of-interest setter, the region-of-interest image and the M-mode image of the region of interest produced by the image producer are displayed on a single screen.

7. The ultrasound diagnostic apparatus according to claim 1,
wherein the region-of-interest image and the whole image including the region of the region-of-interest image are displayed on a single screen in accordance with an input instruction.

8. The ultrasound diagnostic apparatus according to claim 1, further comprising:
an analyzer for analyzing an ultrasound image,
wherein an analysis result by the analyzer is incorporated into the whole image and displayed.

9. The ultrasound diagnostic apparatus according to claim 1,
wherein the whole image is an ultrasound image of the same size as an ultrasound image when the region of interest is set.
